(19) United States
(12) Patent Application Publication
(10) Pub. No.: US 2002/0022832 A1
(43) Pub. Date: Feb. 21, 2002

(54) Cryoprobe assembly with detachable sheath

(76) Inventors: Paul W. Mikus, Irvine, CA (US); Jay J. Eum, Irvine, CA (US)

Correspondence Address: LAWRENCE N. GINSBERG, ENDOCARE, 7 STUDEBAKER, IRVINE, CA, 92618, US

(21) Appl. No.: 09/978,653

(22) Filed: Oct. 16, 2001

Related U.S. Application Data
(63) Continuation-in-part of application No. 09/099,611, filed on Jun. 19, 1998.

(51) Int. Cl. A61B 18/02

(52) U.S. Cl. 606/20; 606/23

(57) ABSTRACT

The cryoprobe assembly includes a cryoprobe and an outer sheath assembly detachably connected thereto. The cryoprobe includes: a Joule-Thomson nozzle; a high pressure gas supply line for supplying gas to the Joule-Thomson nozzle; a heat exchanger interposed between the high pressure gas supply line and the Joule-Thomson nozzle; a cryoprobe sheath containing the heat exchanger and Joule-Thomson nozzle, the cryoprobe sheath having an outer surface; and, a handle attached to the first sheath, the handle having a gripping portion directly graspable by an operator and a connecting portion. The outer sheath assembly includes an adapter covering substantially none of the gripping portion of the cryoprobe handle. The adapter is for attachment to the connecting portion. An outer sheath is connected to the adapter and surrounds the outer surface of the cryoprobe sheath wherein the outer sheath provides enhanced protection against any gas leaks.

### CROSS REFERENCE TO RELATED APPLICATIONS

This application is a continuation-in-part of U.S. Ser. No. 09/099,611 filed on Jun. 19, 1998.

### BACKGROUND OF THE INVENTION

The present invention relates to cryoprobes, and to cryoprobes for use in cryosurgery. In particular, the invention relates to sheathed cryoprobes capable of shaping ice balls formed thereon and to methods of endometrial ablation and other surgical procedures using such cryoprobes.

Cryosurgical probes are used to treat a variety of diseases. The cryosurgical probes quickly freeze diseased body tissue, causing the tissue to die after which it will be absorbed by the body or expelled by the body. Cryothermal treatment is currently used to treat prostate cancer and benign prostate disease, breast tumors and breast cancer, liver tumors and cancer, glaucoma and other eye diseases. Cryosurgery is also proposed for the treatment of a number of other diseases.

The use of cryosurgical probes for cryoablation of the uterus is described in Cahan, W G. and Brockunier, A., Cryosurgery of the Uterine Cavity. Am. Obstet. Gynec. 99:138-153, 1967. Cahan and Brockunier describe a cryosurgical probe patterned after the curve and diameter of a No. 6 Hegar dilator. Liquid nitrogen circulates through this cryosurgical probe in order to cause cryonecrosis of the diseased endometrial tissue in the uterus. Multiple applications of freezing and thawing are applied using the curved probe in order to treat left and right cornu of the uterus as well as the fundus. This method of cryosurgery has a number of drawbacks because the uterus has, for example, an irregular shape resulting from the left and right cornu. Moreover, the uterus has a rough and irregular lining which is not amenable to efficient cryosurgery. Because of the uterus's irregular shape and rough lining, a clinician will often miss a portion of the diseased tissue and must subject the patient to multiple sessions of cryosurgery. A number of approaches have been developed to more efficiently perform cryo-endometrial ablation.

For example, Droegemueller et al., U.S. Pat. No. 3,924,628, disclose a flexible bladder, which is inserted into the uterus. Using a metal catheter, liquid nitrogen is pumped into the bladder that distends to contact the varied surface of the uterine inner lining. However, the bladder is difficult to position properly and may miss portions of diseased tissue.

Coleman et al, U.S. Pat. No. 5,403,309, disclose a cryosurgical probe having a channel for introduction of a heat-conducting liquid into bodily cavities such as the uterus or bladder. A cryoprobe, preferably a Joule-Thompson probe, then cools the heat-conducting liquid to induce cryonecrosis of the diseased tissue. The above methods, however, all suffer from safety problems that are particularly acute for the highly vascular tissue of the uterus. Joule-Thomson probes use high-pressure gas that, should the probe leak, could easily cause gas embolism in such vascular tissue. Thus, there is a need for a cryoprobe providing greater assurance against possible gas leaks. Cryoprobes may be used, as mentioned above, to treat diseases of the prostate, liver, and breast, and they have gynecological applications as well. The cryosurgical probes form ice balls which freeze diseased tissue. Each application has a preferred shape of ice ball, which, if capable of production, would allow cryonecrosis of the diseased tissue without undue destruction of surrounding healthy tissue. For example, prostate cryoablation optimally destroys the lobes of the prostate, while leaving the surrounding neurovascular bundles, bladder neck sphincter and external sphincter undamaged. The prostate is wider at the base and narrow at the apex. A pear or fig shaped ice ball is preferred for this application. Breast tumors tend to be small and spherical so that spherical ice balls are desired to destroy the tumors without destroying surrounding breast tissue. Liver tumors may be larger and of a variety of shapes, including spherical, olive shaped, hot dog shaped or irregularly shaped, and may require more elongated ice balls larger ice balls, and ice balls of various shapes.

U. S. Pat. No. 5,800,487, issued to Mikus et al, the contents of which are incorporated by reference in their entirety as if set forth herein, discloses Joule-Thomson cryoprobes adapted to shape the type of ice ball formed thereon. By varying the length of the heat exchanger coils, the distance between the Joule-Thomson nozzle and the heat exchanger distal end, and the distance between the end of the heat exchange chamber and the Joule-Thomson nozzle, various ice ball shapes are formed. Preferably, a flow-directing sheath is used to further affect the shape of the desired ice ball. Despite the advances set forth by Mikus et al there remains a need in the art for a clinician to have greater control over ice ball shape formation.

### SUMMARY OF THE INVENTION

In one innovative aspect a cryoprobe in accordance with the present invention may comprise a Giaque-Hampson heat exchanger with finned tube gas supply line coiled around a mandrel. The distal portion of the finned tube gas supply line ends in a Joule-Thomson nozzle. An expansion chamber is located distally with respect to the Joule-Thomson nozzles. After exiting the Joule-Thomson nozzles and expanding in the expansion chamber of the cryoprobe, the gas flows over the coils and exhausts out the proximal end of the probe. Proximal to the heat exchanger is a coaxially-disposed insulating layer on the sheath upon which ice formation is curtailed, thereby affecting the shape of the formed ice ball. The insulating layer may be tapered or of a uniform thickness.

In another broad aspect the invention comprises a cryoprobe assembly having a cryoprobe and an outer sheath assembly detachably connected thereto. The cryoprobe includes:

a Joule-Thomson nozzle;

a high pressure gas supply line for supplying gas to the Joule-Thomson nozzle;

a heat exchanger interposed between the high pressure gas supply line and the Joule-Thomson nozzle;

a cryoprobe sheath containing the heat exchanger and Joule-Thomson nozzle, the cryoprobe sheath having an outer surface; and,

a handle attached to the first sheath, the handle having a gripping portion directly graspable by an operator and a connecting portion.

The outer sheath assembly includes an adapter covering substantially none of the gripping portion of the cryoprobe handle. The adapter is for attachment to the connecting portion. An outer sheath is connected to the adapter and surrounds the outer surface of the cryoprobe sheath wherein the outer sheath provides enhanced protection against any gas leaks.

A thermally conducting fluid may be used to fill any space between the outer sheath and the cryoprobe sheath to enhance ice formation. In one embodiment, a channel extends through the adapter to an output port whereby the output port is in fluid communication with the space between the cryoprobe sheath and the outer sheath. A sensor, which can be a pressure transducer or a chemical sensor, is associated with the output port and detects the presence of gas leaks.

### BRIEF DESCRIPTION OF THE DRAWINGS

FIG. 1 is a schematic drawing of a cryoprobe in use during an endometrial ablation procedure.

FIG. 2 is a view of an insulating layer with a single coil Giaque-Hampson heat exchanger according to one embodiment of the invention.

FIG. 3 is a view the distal end of a cryoprobe illustrating an insulating layer with a dual helix heat exchanger according to one embodiment of the invention.

FIG. 4 is a view of a cryoprobe with an outer sheath, adapter, and a pressure sensor port according to one embodiment of the invention.

FIG. 5 is a view of an outer sheath and an adapter according to one embodiment of the invention.

FIG. 6 is a view of a cryoprobe with an outer sheath and adapter removed.

FIG. 7 is a view of an outer sheath and an adapter according to another embodiment of the invention, illustrating the use of a threadably secured adapter.

FIG. 8 is another embodiment in which the outer sheath assembly attachable to the cryoprobe by a snap fit.

FIG. 9 is another embodiment in which the outer sheath assembly attachable to the cryoprobe by a twist and lock assembly.

### DETAILED DESCRIPTION OF THE PREFERRED EMBODIMENTS

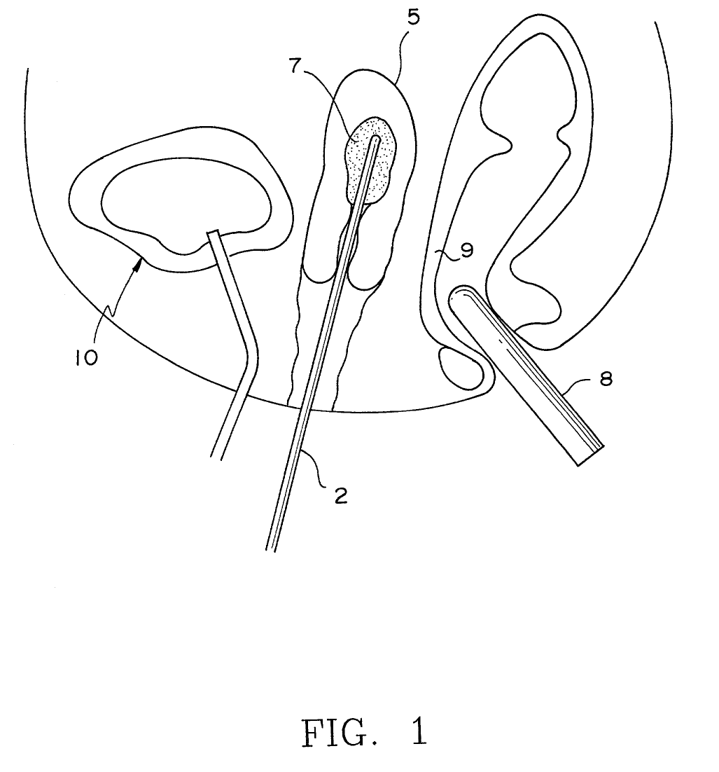

Turning now to the drawings, FIG. 1 shows a cryoprobe being used in an endometrial ablation procedure. A cryoprobe 2 is inserted through the vagina and cervix into the uterus 5. Prior to cryotherapy, the uterus is distended with a heat-conducting fluid 7, preferably 10 cc of sterile intrauterine gel. The bladder 10 is filled with approximately 300 to 400 ml of warm sterile saline to act as heat sink to protect it from cryonecrosis. An ultrasound probe 8 is inserted in the rectum 9 to monitor probe placement and ice ball formation. The cryoprobe 2 is optimally placed in the uterine fundus with the distal tip just touching the uterine wall. A freezing cycle is begun so that a temperature of −40° C. and below is induced in the diseased tissue. Using the transrectal ultrasound, a clinician monitors the radius of the ice ball until it is approximately 25-50% through the myometrium. At this point, the freeze cycle is discontinued and the ice ball allowed to thaw. A second freezing procedure should be conducted in the fundus using this same procedure. If, however, the length of the endometrial cavity is greater than 6 cm., the clinician may dislodge the cryoprobe 2 from the latter formed ice ball when the distal tip temperature reaches 0° and pulls the tip into the lower uterine segment in order to freeze the lower uterine segment. When the formed ice ball encompasses the entire endometrial cavity, thawing is initiated for a second time.

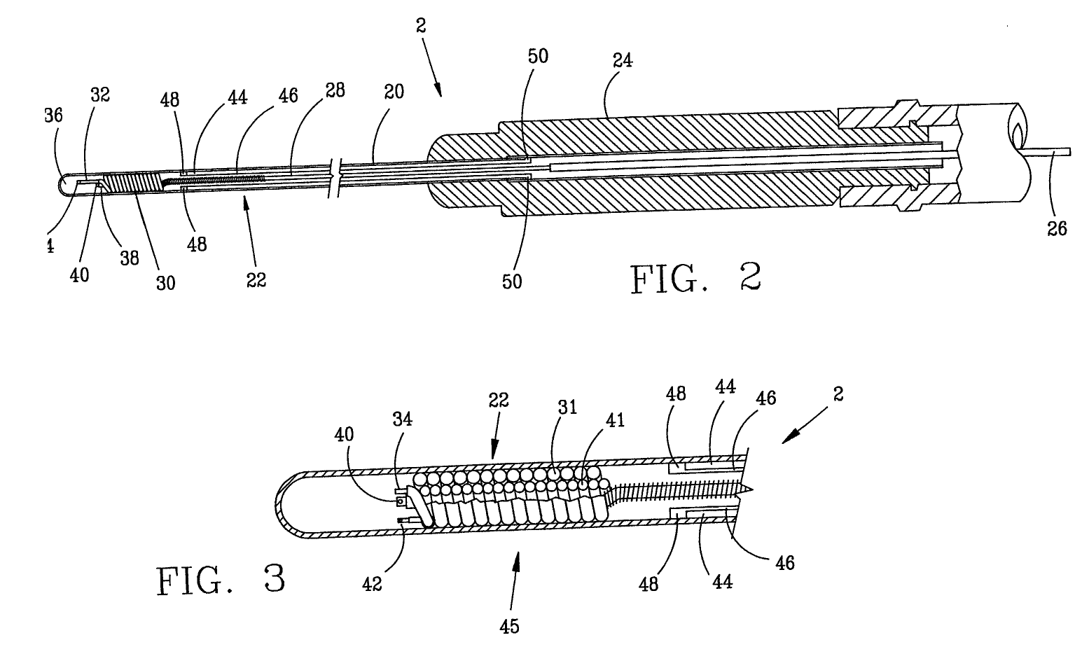

FIG. 2 shows a cryoprobe 2 according to one embodiment of the invention. A first sheath 20 houses the cryostat 22 described in detail below. A handle 24 of convenient size is provided. The handle 24 houses a high pressure gas supply line 26 and electrical wiring (not shown).

The details of the cryostat 22 used in the cryoprobe 2 are illustrated in FIGS. 2 and 3. FIG. 2 shows a first embodiment of the cryoprobe 2. The high-pressure gas supply line 26 connects to the proximal extension 28 of the finned tube coiled heat exchanger 30. The heat exchanger 30 extends longitudinally through the first sheath 20 and connects to the distal extension 32, which opens through Joule-Thomson nozzle 34 into expansion chamber 36. The heat exchanger 30 is coiled around mandrel 38 so that the construction known as a Giaque-Hampson heat exchanger is formed. At the distal tip of the mandrel 38 a thermocouple 40 may be provided so that the clinician can monitor the temperature inside the cryoprobe 2.

FIG. 2 illustrates a single coiled heat exchanger. Alternatively, FIG. 3 shows a dual helixcryoprobe that includes two coiled heat exchangers 41 and 31 and two Joule-Thomson nozzles. This dual helix cryoprobe 45 produces large ice balls. A second high-pressure gas supply line (not illustrated), heat exchanger 41 and Joule-Thomson nozzle 34 are provided. The helical coils preferably are parallel to each other, meaning that the coils follow the same helical path around the mandrel. As shown in FIG. 3, when the Joule-Thomson nozzles 42 and 34 are located at the same longitudinal location, a large spherical ice ball can be formed very rapidly. When the Joule-Thomson nozzles are offset or staggered, meaning that the longitudinal placement of each nozzle is significantly different, the probe very rapidly forms a cylindrical ice ball.

Modifications of the configuration illustrated in FIGS. 2 and 3 will create various ice ball shapes. For convenience of reference, we refer to three longitudinal segments of the cryoprobe as L1, L2, and L3. The distance between the Joule-Thomson nozzle and the end of the heat exchanger is denoted L3. The length of the distal extension 32 is denoted L2. The length of the heat exchanger is denoted LI. With L1 set at approximately 5 cm, if L2 is approximately 7.5 mm and L3 is approximately 5 mm, a pear ice ball shape may be formed. Alternatively, should an olive shaped ice ball be desired, L3 is shortened to approximately 2.5 mm. Although by varying these three parameters, it is possible to form ice balls of various other shapes, an insulating layer 44 provided on the inner surface of the sheath proximal to the heat exchanger affords even greater ice ball shaping control.

FIGS. 2 and 3 illustrate one embodiment of a cryoprobe 2 including the insulating layer 44. A coaxially-disposed inner sheath 46 having a diameter smaller than that of the first sheath 20 forms insulating layer 44. At either end of the inner sheath 44 are distal seal 48 and proximal seal 50 whereby the sheath 20, seals 48 and 50, and inner sheath 46 enclose insulating layer 44. Insulating layer 44 may be comprised simply of air or of another insulating dielectric material. As illustrated in FIGS. 2 and 3, the insulating layer 44 is of a uniform thickness. Alternatively, the insulating layer could be tapered so that the insulation does not begin abruptly at the proximal end of the heat exchanger. This would allow a “feathering” to the proximal edge of the ice ball. Of course, this requires a similar tapering in the diameter of inner sheath 46. By displacing distally or proximally the distal end of the insulating layer 44 as defined by distal seal 48, the ice ball is lengthened or shortened. In addition, the shape of the distal edge of the ice ball may be significantly affected.

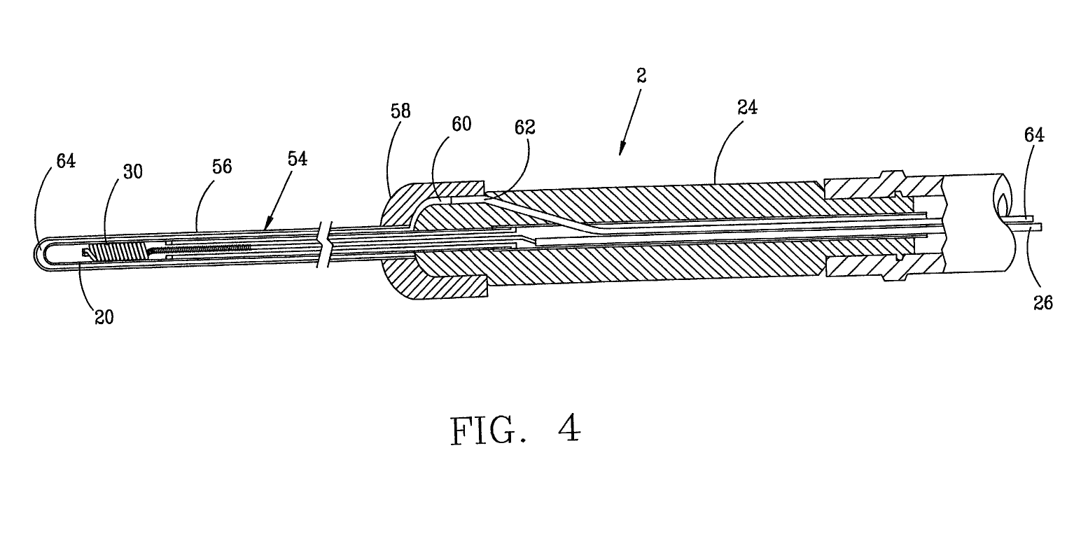

FIG. 4 illustrates an outer sheath 56 and adapter 58 of an outer sheath assembly 54, and pressure sensor port 62 according to one embodiment of the invention. Outer sheath 56 surrounds the cryoprobe sheath 20 so that the danger of gas leaks is lessened. In turn, this greatly reduces the risk of gas embolism causing death or trauma to the patient. The danger of gas embolism is particularly acute during endometrial ablation because of the highly vascular nature of the uterus. Outer sheath 56 and sheath 20 define a space 64. Space 64 can be of negligible thickness or greater provided that thermal conductivity between outer sheath 56 and sheath 30 is not negatively affected to the point that therapeutic efficacy is threatened. Filling space 64 with a petroleum jelly or similar heat-conducting fluid enhances the thermal conductivity of space 56. In addition, outer sheath 56 is preferably made of surgical stainless steel so that its thermal conductivity is high.

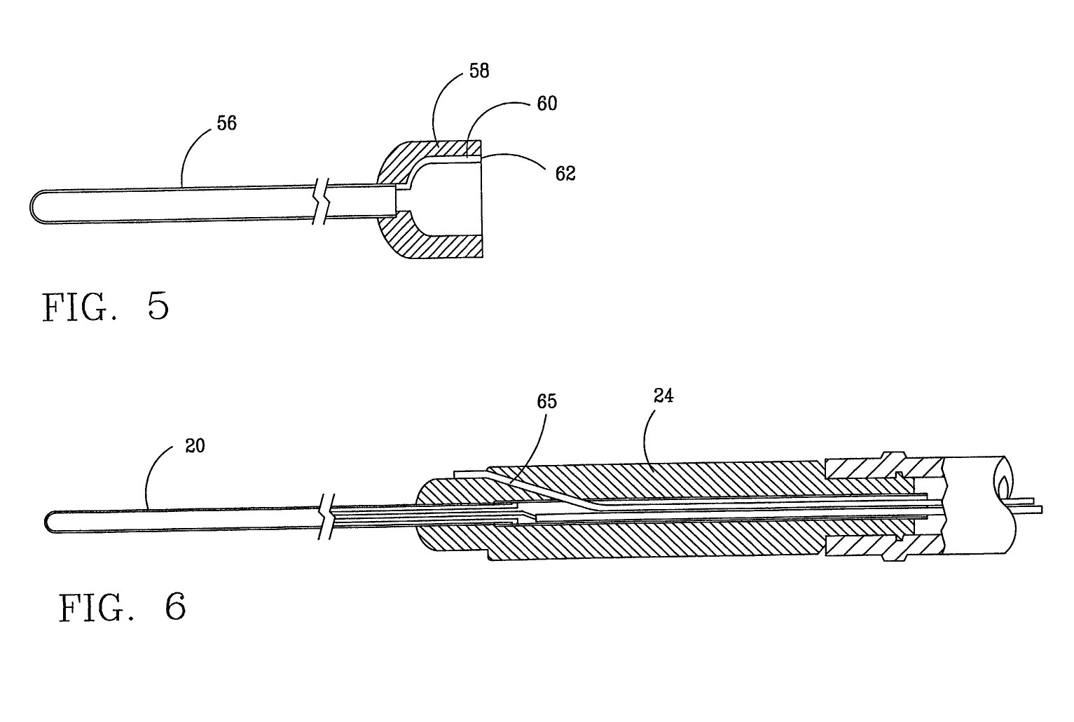

In one embodiment adapter 58 attaches the outer sheath 56 in a sealing arrangement to the handle 24 of the cryoprobe 2. FIG. 5 illustrates the outer sheath 56 and adapter 58 removed from the cryoprobe 2. FIG. 6 illustrates how the handle 24 is machined to fit with adapter 58. Crossing through adapter 58 is a channel 60, which ends in pressure sensor port 62 so that pressure sensor port 62 is in fluid communication with space 64. A pressure sensor tube 65 connects to pressure sensor port 62 so that a sensor (not illustrated) remote from the handle can detect gas leaks. Alternatively, the sensor could be located in the handle 24. The sensor may comprise a pressure transducer or a chemical sensor attuned to a particular gas, preferably argon, used as the high-pressure gas. If the sensor detects a gas leak, pumping of the high-pressure gas could be automatically ceased and an alarm given. The clinician could then remove the cryoprobe before any danger of gas embolism.

In a basic embodiment, the cryoprobe may simply employ an outer sheath 56 and adapter 58 without the channel 60, pressure port 62 and associated sensor. Although there would be no alarm possible in this embodiment, the patient would still enjoy the added security provided by the outer sheath against gas embolism.

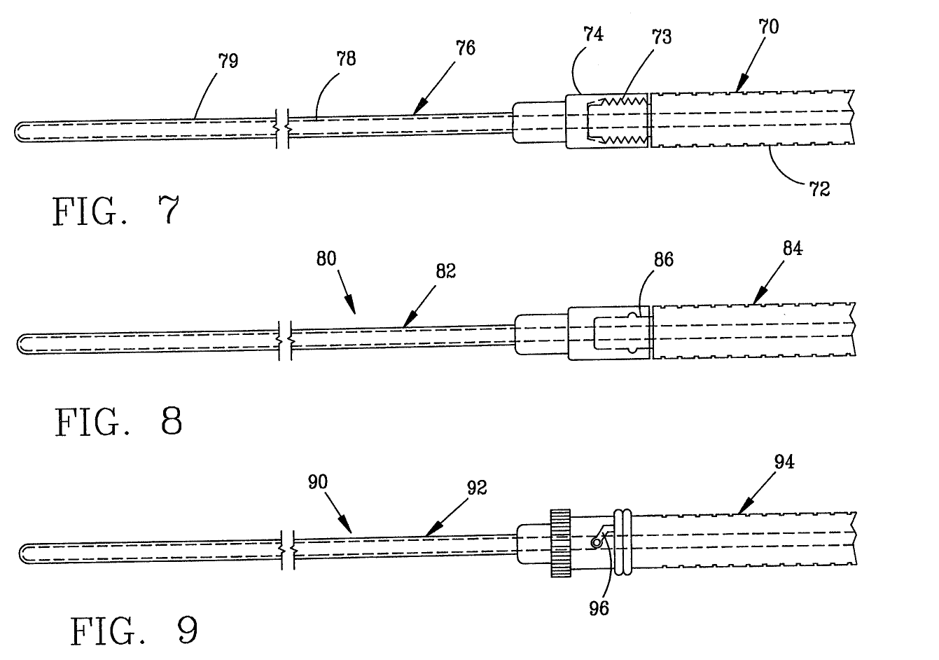

FIGS. 7-9 illustrate alternative means of attaching an outer sheath assembly to the cryoprobe, other than the friction-fit shown with respect to the FIG. 4-6 embodiment. In FIG. 7 the cryoprobe, designated generally as 70, including its handle 72 is threadably attached, as shown by numeral designation 73, to an adapter 74 of an outer sheath assembly, designated generally 76. The cryoprobe sheath 78 of the cryoprobe 70 is surrounded by the outer sheath 79 of the outer sheath assembly 76.

FIG. 8 shows another embodiment of the invention, designated generally as 80, in which the outer sheath assembly 82 is attachable to the cryoprobe 84 via a snap fit 86.

FIG. 9 shows another embodiment of the invention, designated generally as 90, in which the outer sheath assembly 92 is attachable to the cryoprobe 94 via a twist and lock mechanism 96.

Although the use of an outer sheath assembly is particularly useful for endometrial ablation 10 because of the highly vascular nature of the uterus, cryosurgery on other organs in the body will also benefit from the added safety of this invention. Moreover, the benefits provided by the insulating layer whereby the ice ball can lengthened, shortened and distal edge shape affected are not limited to endometrial ablation but can enhance other forms of cryosurgery as well. Thus, while the preferred embodiments of the devices and methods have been described in reference to the environment in which they were developed, they are merely illustrative of the principles of the invention. Other embodiments and configurations may be devised without departing from the spirit of the invention and the scope of the appended claims.

1. A cryoprobe assembly, comprising:
a) a cryoprobe, comprising:
a Joule-Thompson nozzle;
a high pressure gas supply line supplying gas to said Joule-Thomson nozzle;
a heat exchanger interposed between said high pressure gas supply line and said Joule-Thomson nozzle;
a cryoprobe sheath containing said heat exchanger and said Joule-Thomson nozzle, said cryoprobe sheath having an outer surface; and,
a handle attached to said first sheath, said handle having a gripping portion directly graspable by an operator and a connecting portion; and,
b) an outer sheath assembly, detachably connectable to said cryoprobe, comprising:
an adapter covering substantially none of said gripping portion of the cryoprobe handle, said adapter for attachment to said connecting portion; and,
an outer sheath connected to said adapter and surrounding the outer surface of said cryoprobe sheath wherein said outer sheath provides enhanced protection against any gas leaks.

2. The cryoprobe assembly of claim 1, wherein said cryoprobe and said outer sheath assembly are detachably connectable by a friction fit.

3. The cryoprobe assembly of claim 1, wherein said cryoprobe and said outer sheath assembly are detachably connectable by means of threads.

4. The cryoprobe assembly of claim 1, wherein said cryoprobe and said outer sheath assembly are detachably connectable by means of a snap fit.

5. The cryoprobe assembly of claim 1, wherein said cryoprobe and said outer sheath assembly are detachably connectable by means of a twist and lock mechanism.

6. The cryoprobe assembly of claim 1, wherein said adapter defines a channel, said channel having a proximal end connected to a pressure sensor port whereby said pressure sensor port is in fluid communication with a space defined between said cryoprobe sheath and said outer sheath.

7. The cryoprobe assembly of claim 6, wherein a sensor is in fluid communication with said pressure sensor port.

8. The cryoprobe assembly of claim 7, wherein said sensor comprises a pressure transducer.

9. The cryoprobe assembly of claim 7, wherein said sensor comprises a chemical sensor.

10. The cryoprobe assembly of claim 7, wherein said sensor activates an alarm if a gas leak is sensed.

11. The cryoprobe assembly of claim 1, wherein a space defined between said cryoprobe sheath and said outer sheath is filled with a thermally conductive fluid.
